(19) United States
(12) Patent Application Publication
(10) Pub. No.: US 2015/0315263 A1
(43) Pub. Date: Nov. 5, 2015

(54) MONOLITH-BASED PSEUDO-BIOAFFINITY PURIFICATION METHODS FOR FACTOR VIII AND APPLICATIONS THEREOF

(71) Applicant: CENTRE FOR BIOSEPARATION TECHNOLOGY-VIT, Vellore, Tamil Nadu (IN)

(72) Inventors: Vignesh Narasimhan Janakiraman, Vellore, Tamil Nadu (IN); Rajaasekar Rajagopal Prasanna, Vellore, Tamil Nadu (IN); Agamudi Sivasankaran Kamalanathan, Vellore, Tamil Nadu (IN); Mookambeswaran Arunachalam Vijayalakshmi, Vellore, Tamil Nadu (IN)

(73) Assignee: Centre for Bioseparation Technology - VIT, Vellore (IN)

(21) Appl. No.: 14/648,682

(22) PCT Filed: Nov. 27, 2013
(86) PCT No.: PCT/IB2013/060438

§ 371 Date: May 29, 2015

(30) Foreign Application Priority Data
Nov. 30, 2012 (IN) 5018/CHE/2012

(51) Int. Cl. C07K 14/755; C07K 1/22

(52) U.S. Cl. CPC C07K 14/755; C07K 1/22

(57) ABSTRACT

The present disclosure relates to purification of Factor VIII protein and/or its fragments from various sources by employing monolith based pseudobioaffinity purification methods. In particular, L-histidine over CIM monolith [Histidine Ligand Affinity Chromatography (HLAC)] is used for the purification of wild-type factor VIII from plasma cryoprecipitate, recombinant B-domain deleted factor VIII (rBDD-FVIII) expressed in various host systems and recombinant factor VIII light chain expressed in Pichia pastoris. Further, immobilized metal over CIM monolith [Immobilized metal-ion affinity chromatography (IMAC)] is employed for the purification of wild-type factor VIII, rBDD-FVIII expressed in various host systems and recombinant factor VIII heavy chain expressed in Pichia pastoris. The purification efficiency showcased by the purification methods of the present disclosure is far superior when compared to the presently available methods for the purification of factor VIII which lead to significantly improved results in therapeutic applications involving factor VIII.

### TECHNICAL FIELD

The present disclosure relates to purification of Factor VIII protein and/or its fragments from various sources by employing monolith based pseudobioaffinity purification methods. In particular, L-histidine over CIM monolith [i.e. Histidine Ligand Affinity Chromatography (HLAC)] and immobilized metal over CIM monolith [Immobilized metal-ion affinity chromatography (IMAC)] for the purification of factor VIII and/or its fragments from various sources is described.

### BACKGROUND OF THE DISCLOSURE

Factor VIII:C (FVIII:C) is an essential blood coagulation factor which plays a key role in the pathology of haemophilia. FVIII:C is a plasma protein essential for blood coagulation whose deficiency or defective formation results in the blood clotting disorder known as Haemophilia A. FVIII:C is synthesized as a 300-kd precursor protein comprising of six domains: A1, A2, B, A3, C1 and C2. In the plasma, it circulates as a heterodimer consisting of the heavy chain (A1-A2-B domains) and light chain (A3-C1-C2 domains) as a result of limited proteolysis by thrombin. FVIII:C is activated by further proteolysis of the heavy chain resulting in a heterotrimer consisting of A1, A2 and A3-C1-C2 subunits, which in turn activates downstream zymogens completing the coagulation cascade resulting in a clot.

To counter the loss of factor VIII:C activity observed in haemophilia patients, factor VIII:C is administered as plasma concentrates or as recombinant factor VIII:C expressed using mammalian cells. However, the key difficulty associated with both these methods is the efficient purification and recovery of factor VIII:C from their respective sources.

Classical affinity-based purification methods using antibodies specific to the factor VIII:C molecule are conventionally employed for FVIII:C purification. However, said methods often results in loss of activity due to the harsh elution conditions employed which leads to structural variations in factor VIII:C. It is well known in the art that in order to carry out functional studies and for practical applications, the expressed factor VIII:C protein needs to be purified without significant loss of its activity. But, the currently employed methods for FVIII:C purification have several drawbacks which lead to the loss of FVIII:C activity.

The replacement therapy for haemophilia patients using plasma concentrates of factor VIII:C or recombinant factor VIII:C which are currently employed to counter/treat haemophilia A requires improvement in terms of better purity and efficient recovery of factor VIII:C. Such efficient purification and recovery of factor VIII:C translates to money and lives saved. However, as described above, the conventional purification of factor VIII:C involve affinity-based methods which employ harsh elution conditions which may alter the structure of the protein thereby reducing biological activity.

Thus, it can be observed that the presently employed FVIII purification methods are associated with various drawbacks and hence, there is an immense need to arrive at better purification methods for purifying FVIII protein from various sources.

The present disclosure aims at overcoming all the aforesaid drawbacks of the prior art and providing efficient purification methods for Factor VIII.

### STATEMENT OF DISCLOSURE

Accordingly, the present disclosure relates to a method of purifying Factor VIII protein or fragment thereof from a sample, said method comprising act of subjecting the sample to monolith based pseudo-bioaffinity purification to obtain said purified Factor VIII protein or fragment thereof; and a purified, biologically active Factor VIII protein or fragment thereof obtained by the above method, wherein the method purifies Factor VIII protein or fragment thereof with purification factor value ranging from about 30 fold to about 6179 fold.

### BRIEF DESCRIPTION OF ACCOMPANYING FIGURES

In order that the invention may be readily understood and put into practical effect, reference will now be made to exemplary embodiments as illustrated with reference to the accompanying figures. The figures together with a detailed description below, are incorporated in and form part of the specification, and serve to further illustrate the various embodiments, principles and advantages, in accordance with the present disclosure where:

FIG. 1 depicts the chromatography results of plasma cryoprecipitate over CIM-EDA-L-Histidine (i.e. HLAC purification method). Chromatogram (A), 8% SDS-PAGE analysis under non-reducing conditions (B) and Western blotting results (C) are depicted. Different lanes of the SDS-PAGE gel (B) and Western blot (C) correspond to—plasma cryoprecipitate (C), dialysed cryoprecipitate at pHs 7.0 and 6.0 (CD, CD₆), load (L), unbound fraction (Flow-through, FT) and eluted fractions (A₁₀, A₁₁). Pure factor VIII:C is used as control (+). The Mol. Wt. marker used in the gel is a medium-range protein marker. The Mol. Wt. of full length FVIII is about 220 kDa. Arrows on the nitrocellulose membrane (FIG. 1C—Western Blot) indicate the presence of FVIII protein.

FIG. 2 depicts the chromatography of plasma cryoprecipitate over CIM-EDA-Histidine (HLAC) after performing an intermediary wash step. Chromatogram (A) and 8% SDS-PAGE analysis under non-reducing conditions (B) is depicted. Different lanes of the SDS-PAGE gel (B) correspond to plasma cryoprecipitate (C), load (L), unbound fractions (A₁, A₂), elution at 50% elution buffer (A₁₀, A₁₁) and 100% elution buffer (B₉, B₈).

FIG. 3 depicts chromatography of Factor VIII:C light chain over CIM-EDA-Histidine (HLAC). Chromatogram (A) and 10% SDS-PAGE (B) & western blot (C) analysis under non-reducing conditions are depicted. Different lanes of the SDS-PAGE gel (B) and western blot (C) highlight the Load (L), Unbound fractions (1, 2) and Eluted fractions (3, 4), in addition to commercial purified Factor VIII:C (+, used as positive control) and a control Pichia broth (Co) not expressing the light chain protein. Further, FIG. 3(D) depicts the ELISA results performed on various fractions.

FIG. 4 depicts the purification results of heavy chain FVIII:C over CIM-IDA-Cu (i.e. IMAC). The figure showcases chromatogram (A), SDS-PAGE and Western Blotting analysis under non-reducing conditions (B) and SDS-PAGE under reducing conditions (C). Further, the lanes of 4 (B) correspond to control Pichia broth which doesn't express heavy chain (−), load (L), unbound fractions (flowthrough) (F, F′), bound fractions eluted at pHs 6.0/5.0/4.0 (6,5,4) and EDTA-eluted fraction (E). The FVIII:C heavy chain is obtained to near homogenity upon elution at pH 5.0.

FIG. 5 depicts the HLAC purification results of recombinant BDD-FVIII expressed in CHO cells. The figure showcases chromatographic profile without wash step (A) and with wash step (B).

### DETAILED DESCRIPTION OF THE DISCLOSURE

The present disclosure relates to a method of purifying Factor VIII protein or fragment thereof from a sample, said method comprising act of subjecting the sample to monolith based pseudo-bioaffinity purification to obtain said purified Factor VIII protein or fragment thereof.

In an embodiment of the present disclosure, the Factor VIII protein or fragment thereof within the sample is naturally occurring or recombinant Factor VIII protein or fragment thereof, or a combination thereof.

In another embodiment of the present disclosure, the natural Factor VIII protein or fragment thereof is obtained from a source selected from a group comprising plasma and liver or a combination thereof, and the recombinant Factor VIII protein or fragment thereof is obtained from a cell selected from a group comprising CHO cell, Pichia pastoris, Lemna gibba and Hansunella polymorpha, or any combination thereof.

In yet another embodiment of the present disclosure, the Factor VIII fragment is Factor VIII Light Chain, Factor VIII heavy chain, or a combination thereof.

In still another embodiment of the present disclosure, the Factor VIII heavy chain has a molecular weight ranging from about 90 kDa to 210 kDa; and wherein light chain has a molecular weight of about 80 kDa.

In still another embodiment of the present disclosure, the method purifies Factor VIII protein or fragment thereof with purification factor value ranging from about 295 fold to about 6179 fold.

In still another embodiment of the present disclosure, the yield of purified Factor VIII protein or fragment thereof ranges from about 15% to about 55%.

In still another embodiment of the present disclosure, the monolith based pseudo-bioaffinity purification comprises acts of:
a) coupling or immobilizing a monolith column with a ligand to obtain an immobilized monolith column;
b) equilibrating the immobilized monolith column obtained above and loading the sample into the column;
c) optionally carrying out a wash step; and
d) eluting the sample to obtain the Factor VIII protein or fragment thereof.

In still another embodiment of the present disclosure, the ligand is selected from L-histidine or transition metal ion, or a combination thereof.

In still another embodiment of the present disclosure, the monolith based pseudo-bioaffinity purification is Histidine Ligand Affinity Chromatography (HLAC) when the ligand is L-histidine, and wherein the monolith based pseudo-bioaffinity purification is Immobilized metal-ion affinity chromatography (IMAC) when the ligand is transition metal ion.

In still another embodiment of the present disclosure, the transition metal ion is selected from a group comprising copper, nickel, cobalt and zinc, or any combination thereof.

In still another embodiment of the present disclosure, the monolith column is Convective Interaction Media (CIM) monolithic column.

In still another embodiment of the present disclosure, the coupling or immobilization is carried out in presence of coupling agent selected from a group comprising ethylene-di-amine (EDA), carbonyldiimidazole (CDI), epoxy, imino-di-acetic acid (IDA) and Tris(2-aminoethyl)amine (TREN), or any combination thereof.

In still another embodiment of the present disclosure, the equilibration is carried out by employing buffer selected from a group comprising cationic buffer, phosphate buffer, 3-(N-morpholino)propanesulfonic acid (MOPS) buffer and MMA buffer, or any combination thereof, and wherein the phosphate buffer, MOPS buffer and MMA buffer contain salt at concentration ranging from about 0.5 M to about 2 M, preferably sodium chloride at concentration of about 0.5 M.

In still another embodiment of the present disclosure, concentration of the aforesaid cationic buffer ranges from about 20 mM to about 100 mM and pH ranges from about 5.5 to about 6.5, and wherein concentration of the aforesaid phosphate buffer, MOPS buffer and MMA buffer ranges from about 20 mM to about 100 mM and pH ranges from about 7.0 to about 8.0.

In still another embodiment of the present disclosure, the elution is carried out by employing buffer selected from a group comprising cationic buffer containing calcium (II) ions, glycinate ions and lysine, acetate buffer containing sodium chloride, or a combination thereof.

In still another embodiment of the present disclosure, concentration of the cationic buffer as mentioned for elution ranges from about 20 mM to about 100 mM and pH ranges from about 7 to about 8, and wherein concentration of the acetate buffer ranges from about 20 mM to about 100 mM and pH ranges from about 4 to about 5.

In still another embodiment of the present disclosure, the wash step is carried out by employing buffer selected from a group comprising cationic buffer, phosphate buffer, 3-(N-morpholino)propanesulfonic acid (MOPS) buffer and MMA buffer, or a combination thereof.

In still another embodiment of the present disclosure, concentration of the cationic buffer as mentioned for wash step ranges from about 10 mM to about 50 mM and pH ranges from about 7 to about 8, and wherein concentration of the phosphate buffer, 3-(N-morpholino)propanesulfonic acid (MOPS) buffer and MMA buffer ranges from about 20 mM to about 100 mM and pH ranges from about 6.0 to about 7.0.

In still another embodiment of the present disclosure, the cationic buffer as mentioned above for equilibration, elution and wash steps is selected from a group comprising Tris-HCl, and bicarbonate, or a combination thereof.

The present disclosure further relates to a purified, biologically active Factor VIII protein or fragment thereof obtained by the above method, wherein the method purifies Factor VIII protein or fragment thereof with purification factor value ranging from about 30 fold to about 6179 fold.

Pseudobioaffinity-based purification methods are used to obtain proteins of our interest by employing mild elution conditions, thus avoiding protein denaturation. Said pseudo-bioaffinity based purification methods involve interaction between proteins and immobilized metals or proteins and histidine which are made specific under optimal conditions to enable single-step purification of proteins from biological mixtures.

In the present disclosure, two pseudobioaffinity-based purification methods namely, Histidine Ligand Affinity Chromatography (HLAC) and Immobilized Metal Affinity Chromatography (IMAC) are employed for purification of FVIII protein. HLAC is used for the purification of factor VIII:C from various sources such as plasma cryoprecipitate, recombinant B-domain deleted factor VIII:C expressed in CHO cell lines and recombinant factor VIII:C light chain expressed in Pichia pastoris. Similarly, IMAC is employed for the purification of recombinant factor VIII:C from several sources such as plasma cryoprecipitate, recombinant B-domain deleted factor VIII:C expressed in CHO cell lines and recombinant factor VIII:C heavy chain expressed in Pichia pastoris. In an embodiment, the molecular weight of full-length FVIII ranges from about 170 kDa to about 290 kDa, depending on the extent of proteolysis of the heavy chain. Further, the molecular weight of BDD-FVIII used in the present disclosure has a molecular weight of about 170 kDa.

Histidine ligand affinity chromatography (HLAC), is employed by the present disclosure to retain Factor VIII and/or its fragments (preferably, Factor VIII-Light Chain) with high selectivity, based on a combined hydrophobic and ion pairing complementarity. This high selectivity is possible due to the uniqueness of the amino acid L-Histidine: its mild hydrophobicity, weak charge transfer possibilities and wide pKa range. These factors result in a multimodal interaction of weak to average binding strength, which would in-turn facilitate mild adsorption and elution conditions ensuring structural integrity of the Factor VIII and/or its fragments.

The present disclosure successfully employs pseudo-bioaffinity based purification methods involving the amino acid L-histidine as ligand (i.e. HLAC) over CIM monolith and immobilized metal ions (i.e. IMAC) over CIM monolith to efficiently recover factor VIII:C and/or its fragments from various sources using minimal steps, preferably in one step. L-Histidine as ligand coupled to a monolith-based Convective Interaction Media (CIM) support system (CIM-HLAC) is employed to purify factor VIII:C from plasma and various other sources as described above. The CIM stationary phase pose several advantages over other conventional support systems, as they exhibit high dynamic binding capacity for large molecules, flow independent performance and low pressure drop even at high flow rates which enable rapid separation. CIM monoliths are made of monolithic polymethacrylate polymers. These CIM monoliths, or Short Monolithic Columns (SMCs) are prepared by free radical polymerization of a mixture of glycidyl methacrylate (providing functional groups), ethylene dimethacrylate (as a cross-linking reagent), 2,2′-azobisisobutyronitrile (as an initiator) and a porogenic solvent (cyclohexanol and dodecanol) in barrels of polypropylene syringes, yielding glycidyl methacrylate-co-ethylene dimethacrylate (GMA-EDMA) monoliths. After polymerization, the block of polymer formed in disk or tube shape, is mounted in a specially designed housing allowing good sample distribution and low dead volume.

Table 1 highlights some of the advantages of CIM monolithic supports over conventionally packed columns.

TABLE 1
Comparison of monolithic supports
with conventionally packed columns

| Chromato- | | | | Range of |
|---|---|---|---|---|
| graphic | Nature of | Binding | Scale | accepted |
| Support | flow | efficiency | up | proteins |
| Conventionally | Diffusion- | Restricted | Difficult | Restricted |
| packed | based | to low flow | | by pore size |
| columns | | rates due to | | |
| | | back | | |
| | | pressure | | |
| Monoliths | Convective | Independent | Easy | Accommodates |
| | | of flow rates | | large proteins |

Thus, by combining the ultra-fast flow system of CIM with pseudobioaffinity based ligand L-Histidine or immobilized metal ions, the present disclosure provides for quick and efficient purification of factor VIII and/or its fragments from various sources.

In an embodiment of the present disclosure, Histidine Ligand Affinity Chromatography (HLAC) over CIM support is used for the rapid purification of FVIII:C from plasma.

In another embodiment of the present disclosure, Histidine Ligand Affinity Chromatography (HLAC) over CIM support is used for the rapid purification of recombinantly expressed FVIII light chain (FVIII-LC).

In yet another embodiment of the present disclosure, HLAC over CIM support is used for the rapid purification of recombinantly expressed B-domain deleted (BDD)/full-length FVIII from various host systems not limited to CHO cell lines, Pichia pastoris, Lemna gibba and Hansenula polymorpha.

In an exemplary embodiment of the instant disclosure, the purification of FVIII-LC or BDD/full length FVIII by HLAC occurs by the binding of the ligand L-Histidine to the light chain of FVIII or BDD/full length FVIII wherein, the binding mechanism of BDD/full length FVIII protein to L-Histidine is through the light chain. L-Histidine interacts with BDD/full length FVIII in a multi-modal manner, owing to its unique properties: wide pKa range, mild hydrophobicity and weak charge transfer possibilities. Binding of FVIII to L-Histidine is favoured under mild acidic conditions, and elution is achieved upon raising the pH to near-neutral levels along with the addition of calcium (II) ions, which stabilizes the FVIII molecule. While the light chain of factor VIII is expected to bind to L-Histidine by both positive and negative ionic forces, the retention of L-Histidine is primarily through positive forces of binding based on surface exposed charges. The addition of lysine in the elution buffer helps break these specific ionic bonds.

In yet another embodiment of the present disclosure, Immobilized Metal-ion Affinity Chromatography (IMAC) over CIM monoliths is employed to rapidly purify factor VIII protein and/or its fragments from various natural and recombinant sources. In an exemplary embodiment, IMAC is employed to purify recombinantly expressed heavy chain of factor VIII:C. The rationale behind employing IMAC in the present disclosure is based upon the examination of the 3-dimentional structure of the heavy chain of factor VIII:C which reveals 3-4 Histidine residues exposed on the surface of the heavy chain. The principle of metal chelating with the exposed surface having histidine is employed here, without the need for any additional affinity tags. In other words, the immobilised metal affinity chromatography (IMAC) technique takes advantage of the property of certain amino acids like Histidine to bind to chelated transition divalent metal ions (such as copper, nickel, cobalt, zinc or iron). This binding is facilitated at near neutral pH, wherein the deprotonated imidazole ring of the exposed Histidine residues form coordinate bond with the immobilized metal. Lowering the pH of the buffer results in protonation of imidazole, leading to a break in the coordinate bond subsequently elution of the protein.

Therefore, the present disclosure successfully employs pseudo-bioaffinity based purification methods involving the amino acid L-histidine as ligand (i.e. HLAC) over CIM monolith and immobilized metal ions (i.e. IMAC) over CIM monolith to rapidly and efficiently purify factor VIII:C and/or its fragments from various sources using minimal steps.

Additional embodiments and features of the present disclosure will be apparent to one of ordinary skill in art based upon description provided herein. However, the examples and the figures should not be construed to limit the scope of the present disclosure.

Materials Used for Arriving at the Examples of the Present Disclosure

Chromatographic procedures involving plasma-factor VIII:C and recombinant factor VIII:C light chain are carried out using CIM (Convective Interaction Media) monolithic disk (BIA separations d.o.o., Slovenia), coupled with L-Histidine as ligand using a new coupling chemistry. Chromatographic procedures involving recombinant factor VIII:C heavy chain are carried out over CIM-IDA monolithic disk chelated with Cu⁺⁺ ions (ligand). Protein samples obtained from chromatographic procedures are concentrated using Amicon® Ultra-4 Centrifugal Filter Units (Millipore, Billerica, USA). Buffers for chromatographic runs and reagents are prepared using chemicals of analytical grade either from Sigma-Aldrich (St. Louis, USA) or HiMedia (Bangalore, India).

Protein purification (chromatographic procedures) is performed using ÄKTA FPLC workstation from GE Healthcare and is monitored with Unicorn 5.1 software (GE Healthcare, Uppsala, Sweden). Protein detection is monitored at 280nm. Further, spectrophotometric measurements of samples are made using Beckman Coulter DU730 UV-Vis Spectrophotometer (Beckman Coulter, Hope City, USA).

Plasma cryoprecipitate for purification of factor VIII:C is obtained from Department of Haematology, Christian Medical College (CMC), Vellore. The recombinant factor VIII:C heavy chain and light chains are expressed in house using the yeast expression host Pichia pastoris GS115.

### EXAMPLE 1

Purification of Factor VIII:C from Plasma

Plasma can be employed as a direct natural source for obtaining FVIII protein. Alternatively, plasma cryoprecipitate can be prepared and used as a source of FVIII.

In a specific embodiment, the plasma cryoprecipitate [which is the precipitate obtained by the freezing of fresh-frozen plasma followed by its thawing at 4° C.] is pre-treated by dialysing the said cryoprecipitate against 20 mM Tris, pH 7.0 over a period of about 6 hours at about 4° C. The pH of the dialysed sample is reduced using 20 mM Tris at pH 6.0 which results in the formation of a precipitate. The sample is centrifuged at 10,000 g for about 5 minutes, and the supernatant is used as source material for the chromatographic run.

Purification of factor VIII:C from dialysed plasma cryoprecipitate is carried out by HLAC using the amino acid L-Histidine as ligand. L-Histidine can be immobilized/coupled to CIM support through a number of coupling groups, including expoxy, ethylene-di-amine (EDA) and carbonyldiimidazole (CDI) chemistries. The inventors of the present disclosure have successfully coupled L-Histidine through all three chemistries, wherein the coupling/immobilization using EDA chemistry is described in detail. A fresh CIM-EDA disk is equilibrated with 50 mM Phosphate buffer, pH 7.5 at a flow rate of about lml/min for about 1 hour at room temperature. About 30 ml of 10% glutaraldehyde solution (v/v) in 50 mM phosphate buffer, pH 7.5 is pumped through the disk, after which the disk is incubated with about 5 ml of the same glutaraldehyde solution overnight in dark at about 25° C. for derivatization in a glass beaker. Following derivatization, the disk is washed with about 30 ml of 0.5M phosphate buffer pH 7.5, followed by a second wash with 0.5M phosphate buffer, pH 3.0 for about 1 hour at a flow rate of about lml/min. About 20 ml of L-Histidine solution (about 50 mg/ml L-Histidine monohydrochloride monohydrate in 0.5M phosphate buffer, pH 3.0) is then pumped through the column at aflow rate of about 0.4 ml/min for about 6 hours under recirculation, after which the disk is incubated in the L-Histidine solution in a glass beaker for about 24 hours at 25° C. under constant gentle shaking (about 75-80 rpm). The disk is then washed with about 10 ml of 0.5M phosphate buffer, pH 3.0 followed by about 10 ml of 0.1M cyanoborohydride solution which is prepared by dissolving in 0.5M phosphate buffer, pH 3.0. The disk is thereafter incubated with about 10 ml of the same cyanoborhydride solution for about 3 hours at 25° C. under gentle shaking, after which the excess cyanoborohydride is washed off with 0.5M phosphate buffer pH 3.0. Endcapping or blocking of residual reactive groups in the support matrix, is achieved by passing about 10 ml of 1M monoethanolamine in about 50 mM phosphate buffer, pH 7.5 followed by incubation of the disk in about 5 ml of monoethanolamine solution for about 3 hours at 25° C. The disk is finally washed with 50 mM phosphate buffer, pH 7.8 followed by a wash with 1M NaCl in 25 mM Tris-HCl, pH 7.4. This CIM disk immobilized with L-Histidine (CIM-EDA-L-Histidine) is used for further purification experiments.

The prepared CIM-EDA-L-Histidine disk (dimensions: 12 mm×3mm, volume: 0.34 ml) is placed into the housing to obtain a CIM monolithic column. The binding and elution flow rates are varied between a range of about 3 CV/min to about 18 CV/min. The column is equilibrated using 20 mM-100 mM of cationic buffers such as Tris-HCl at pH 5.5 to 6.5, preferably 20 mM Tris buffer at pH 6.0 ±0.2, and the sample is injected. Elution is performed using 20 mM-100 mM of cationic buffers such as Tris-HCl at pH 7.0 to 8.0, in the presence of Calcium (II) ions, Glycinate ions and Lysine. Preferably, elution is performed using an optimized step gradient of 20 mM Tris buffer, pH 7.0 containing 0.1M Glycine, 0.03M Lysine and 0.3M CaCl₂. The purified fractions are further confirmed by SDS-PAGE and western blotting analysis for the protein of interest and are assayed for their biological activity by performing clotting assays.

As described above, SDS-PAGE, followed by western blotting is performed on purified samples to confirm the presence of factor VIII:C protein. About 5 μg of protein sample is loaded on each well of a polyacrylamide gel (8% polyacrylamide gels), and SDS-PAGE is performed to separate them. After electrophoresis, the proteins from the polyacrylamide gel are transferred to a nitrocellulose membrane using Mini Trans-Blot Cell (BIO-RAD, India). Towbin transfer buffer (25 mM Tris, 200 mM glycine, 0.01% SDS, 20% methanol, pH 8.3) is used to carry out the transfer from polyacrylamide gel to the nitrocellulose membrane, at 90V for about 90 minutes at a temperature of about 4° C. The membrane is thereafter washed with PBST (Phosphate Buffered Saline with 0.1% Tween-20), blocked with a blocking buffer (PBST with 5% skimmed milk powder) overnight at a temperature of about 4° C. After blocking, the membrane is incubated with primary antibody (rabbit anti-A₁ or anti-C₂ antiserum) at room temperature for about one hour. The membrane is thereafter washed for about 2-4 times with PBST solution after which it is incubated with secondary antibody (goat anti-rabbit IgG, HRP conjugated) at room temperature for about 1 hour. The membrane is then washed for about 2-4 times with PBST. The membrane is finally developed by diaminobenzidine/H₂O₂ method for detection of HRP.

The rabbit anti-A₁ and anti-C₂ antisera used as above for the detection of the expressed proteins by western blotting are prepared using E. coli expressed heavy and light chain terminal domains Aₗ and C₂ domains respectively, tagged with GST as antigen. Rabbits are immunized with the purified antigens (A₁-GST and C₂-GST). The antiserum generated to one of these two antigens is used to probe the western blots.

Purification Results

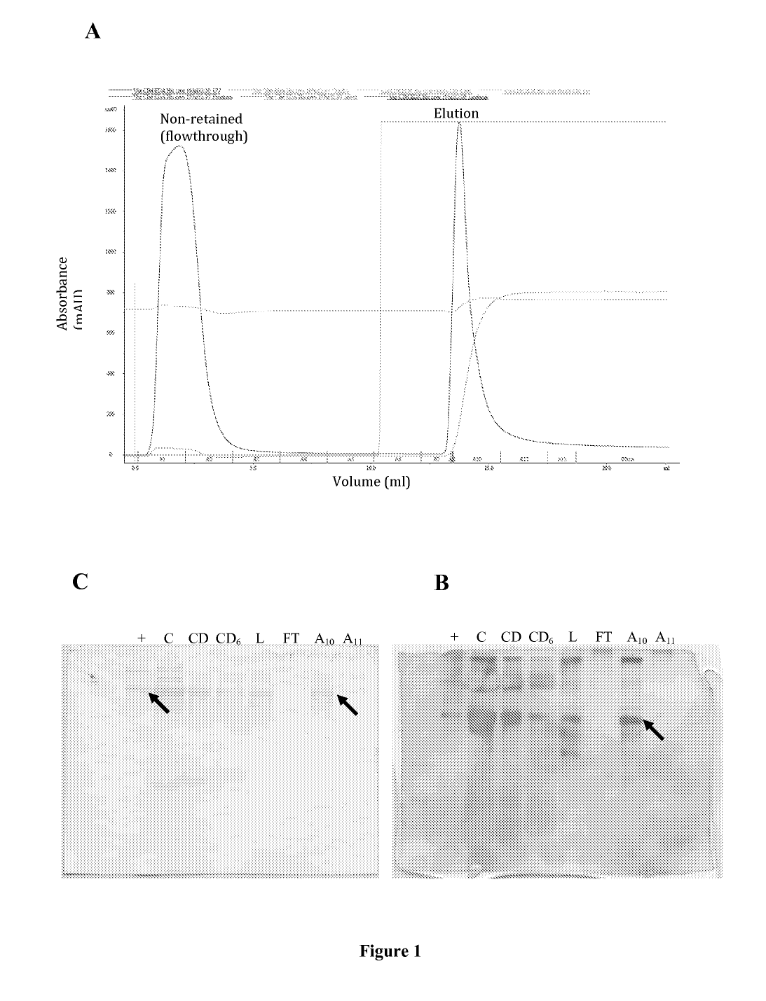

Plasma cryoprecipitate is obtained, pretreated and the sample is injected over an equilibrated CIM-EDA-L-Histidine disk. The chromatogram obtained after HLAC purification is depicted in FIG. 1A. The chromatogram clearly shows the signal corresponding to non-retained proteins flowing through upon sample injections followed by the recovery of factor VIII:C in a sharp peak obtained by passing the elution buffer through the disk. Further, upon SDS-PAGE analysis of the purified fraction (FIG. 1B), a clear band is observed in the tail portion of the eluted fraction (A₁₀), which exhibits substantial coagulation activity verified by clotting assays (Table 2). The purification of Factor VIII:C protein is further confirmed by Western Blot results wherein the A₁₀ fraction clearly shows the presence of Factor VIII:C (FIG. 1C).

Activity Results

The coagulation activity of different fractions during the purification process is measured using one stage clotting assay. The one stage clotting assay is performed as follows

Factor VIII-depleted plasma is dissolved in distilled or deionized water. Before use, it is allowed to stand for at least 15 minutes at a temperature of about 15 to 25° C., and then mixed carefully without foam formation. The sample (one of various fractions, viz., cryoprecipitate, processed cryoprecipitate, load or elute obtained at different stages of the purification process) is added to FVIII deficient plasma. Activated Partial Thromboplastin Time (APTT) reagents are used according to the manufacturer's instructions for determining the coagulation activity. 0.025M of CaCl₂ is added to the mixture. The mixture is incubated at a temperature of about 37° C. for about 2 minutes and the reading is taken in an automated coagulation analyzer. Normal clotting time is 30-40 seconds.

The respective specific activity results are showcased in Table 2 below.

TABLE 2
Activity table for purification of factor VIII:C from plasma
cryoprecipitate

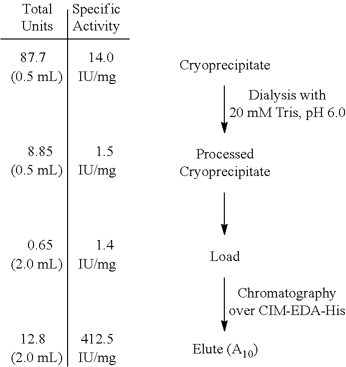

In an embodiment, about 295-fold purification of Factor VIII protein is achieved with a recovery/yield of about 15%. A fraction of the protein is lost in the non-retained fraction (i.e. flow through), which is due to the lower capacity of the CIM-0.34 mL disk. The said protein can be recovered by rechromatographing the flow-through fraction again, or by using a larger disk/column.

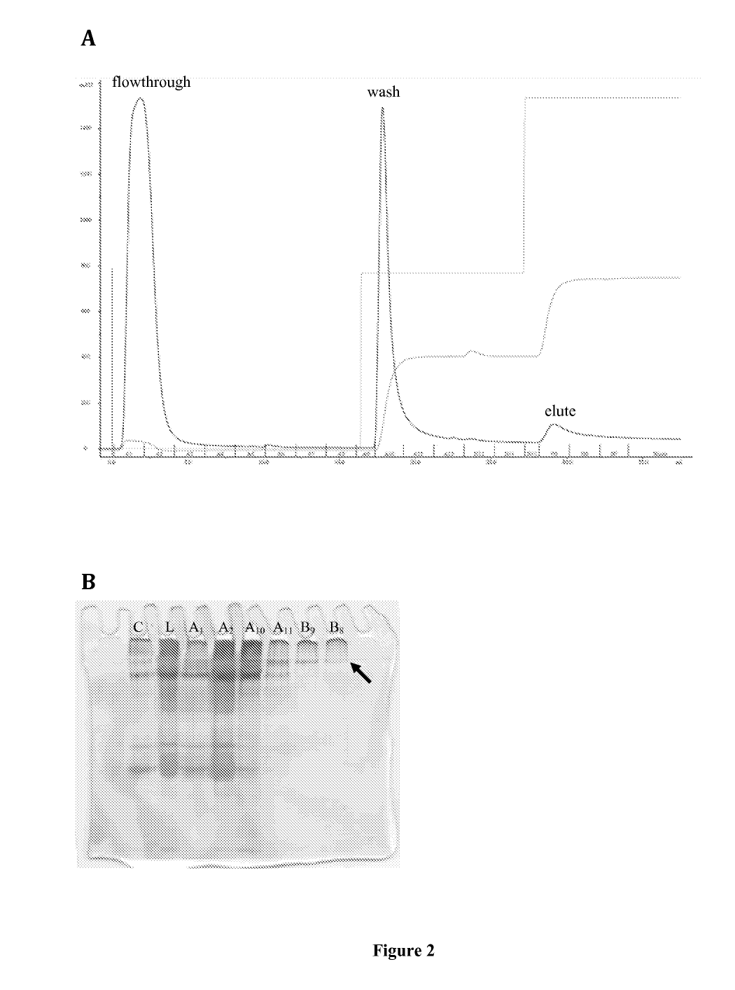

In another embodiment of the present disclosure, the HLAC purification process is further streamlined by the inclusion of an intermediate wash step at 50% to remove the adjoining impurities (FIG. 2). By the introduction of an intermediary wash step using 50% elution buffer, other proteins from the Factor VIII:C mixture gets separated leading to improved purification. In yet another embodiment, the higher purification results achieved due to intermediary wash step is further substantiated by the marked increase in activity exhibited by the eluted fraction B₈ (FIG. 2B and Table 3). The results showcase an increased recovery/yield of 55% and about 1386-fold increase in purity. A fraction of Factor VIII:C protein is lost in the non-retained fraction (flow-through). Hence, the percentage of recovery/yield can be further improved by passing the flow through fraction through the column/disk again, or by using a larger disk/column.

TABLE 3
Activity table for purification of factor VIII:C from plasma
cryoprecipitate (with intermediary wash step)

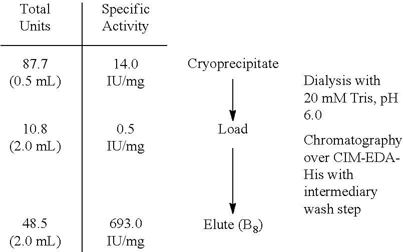

Based on the above results, it is apparent that Factor VIII protein has been purified to high purity from plasma cryoprecipitate using L-Histidine immobilized on CIM support.

### EXAMPLE 2

Purification of Recombinant Factor VIII:C Light Chain

Purification of factor VIII:C light chain (80 kDa) expressed by Pichia pastoris GS115 cells is carried out using CIM-His disk. The CIM-His disk (dimensions: 12 mm×3 mm, volume: 0.34 ml) is prepared as described in the previous example and the same is placed into the housing to obtain a CIM monolithic column. Constant binding and elution flow rates ranging between 3 CV/min to 18 CV/min are employed throughout the experiment. The column is equilibrated using 20 mM-100 mM of cationic buffers such as Tris-HCl at pH 5.5 to 6.5, preferably with 20 mM Tris buffer at pH 6.0, and the sample (Pichia broth expressing factor VIII:C light chain concentrated by 0→80% saturation ammonium sulphate) is injected. Elution is performed in a single step using 20 mM-100 mM of cationic buffers such as Tris-HCl at pH 7.0 to 8.0, in the presence of Calcium (II) ions, Glycinate ions and Lysine. Preferably, elution is performed using 20 mM Tris buffer, pH 7.0 containing 0.1M Glycine, 0.03M Lysine and 0.3M CaCl₂ to obtain purified fractions.

SDS-PAGE, followed by western blotting is performed on purified samples to confirm the presence of factor VIII:C light chain. About 5 μg of obtained protein sample is loaded on each well of a polyacrylamide gel (10% polyacrylamide gels), and SDS-PAGE is performed. After electrophoresis, the proteins from the polyacrylamide gel are transferred to a nitrocellulose membrane using Mini Trans-Blot Cell. Towbin transfer buffer (25 mM Tris, 200 mM glycine, 0.01% SDS, 20% methanol, pH 8.3) is used to carry out the transfer from the polyacrylamide gel to the nitrocellulose membrane at 90V for about 90 mins at a temperature of about 4° C. The membrane is washed with PBST (Phosphate Buffered Saline with 0.1% Tween-20), blocked with a blocking buffer (PBST with 5% skimmed milk powder) overnight at a temperature of about 4° C. After blocking, the membrane is incubated with primary antibody (rabbit anti-A₁ or anti-C₂ antiserum, along with excess of control Pichia broth to prevent non-specific binding) at room temperature for about one hour. The membrane is washed 2-4 times with PBST (Phosphate Buffered Saline solution with the detergent Tween 20) solution after which it is incubated with secondary antibody (goat anti-rabbit IgG, HRP conjugated) at room temperature (25° C.-30° C.) for about 1 hour. The membrane is thereafter washed 2-4 times with PBST. Finally, the membrane is developed by diaminobenzidine/H₂O₂ method for detection of HRP.

The rabbit anti-C₂ antiserum is used to follow the purification of the factor VIII:C light chain protein by western blotting is prepared using E. coli expressed light chain terminal domain C₂ domain, tagged with GST, as antigen. Rabbits are immunized with the purified antigen (C₂-GST). During addition of rabbit antisera to the membrane, the antiserum is incubated along with excess of Pichia pastoris control broth to reduce non-specific binding and background noise.

Purification Results

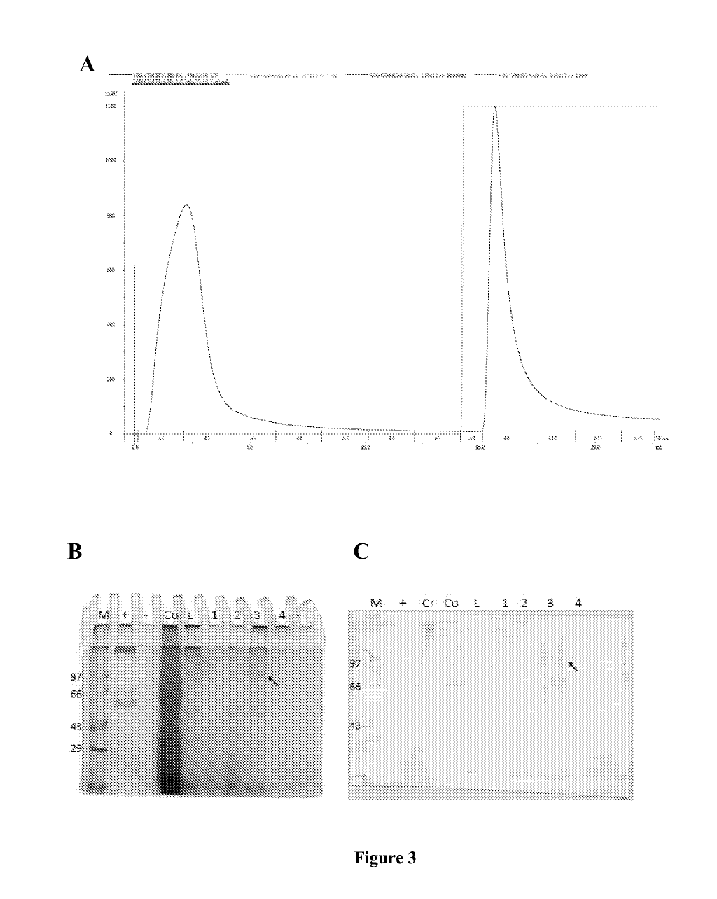

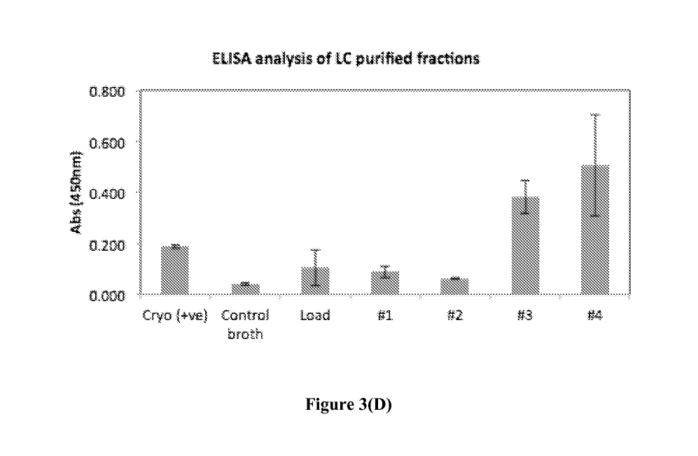

Histidine Ligand Affinity Chromatography (HLAC) is employed to purify the recombinantly expressed light chain of FVIII:C in Pichia pastoris as described above. The load, flow through and eluted fractions of the chromatographic experiment are analyzed over 10% SDS-PAGE under non-reducing conditions. Upon SDS-PAGE and western blotting analysis under non-reducing conditions, a band corresponding to the light chain protein is observed in the eluted fraction (lane 3, FIGS. 3B and 3C). The eluted fraction shows a band slightly above the expected 80 kDa. This increase in the theoretically predicted molecular weight could be attributed to glycosylation on the light chain residues. Nonetheless, the purification and presence of FVIII-Light Chain is successfully confirmed by ELISA analysis [FIG. 3(D)].

This purified factor VIII:C light chain is used for reconstitution with purified heavy chain to exhibit coagulation activity.

### EXAMPLE 3

Purification of Recombinant Factor VIII:C Heavy Chain

Purification of heavy chain of factor VIII:C from the heavy chain expressing Pichia pastoris clone is carried out using immobilized metal ion affinity chromatography (IMAC) using Cu⁺⁺ ions as ligand. The Cu⁻⁺ ions are chelated on to the CIM-IDA [CIM-Imino-Di-Acetic acid] disk (dimensions: 12mm×3 mm, volume: 0.34 ml), and packed into the housing to obtain a CIM monolithic column. Chelation of Cu++ ions on the CIM-IDA disk is achieved by passing about 20 column volumes of 50 mM CuSO₄ over the CIM-IDA disk. Further, the principle of retention of the heavy chain protein by CIM-Cu column is based on the coordinate bond formed between the immobilized divalent transition metal ion (ligand) and the histidine residue of the heavy chain protein. When the pH of the buffer is reduced, the charge on the histidine residue on the heavy chain protein is lost, and hence the coordinate bond is broken and the protein is eluted.

The CIM monolithic column as prepared above is equilibrated with Phosphate/MOPS [3-(N-morpholino)propanesulfonic acid]/MMA buffers [MES-MOPS-Acetate] (concentration ranging between 20 mM-100 mM) containing high salt (0.5M NaCl). Preferably, the column is equilibrated with 50 mM phosphate buffer containing 0.5M NaCl, pH 7.0±0.2. Constant binding and elution flow rates ranging between 3 CV/min to 18 CV/min are employed throughout the experiment. The sample (Pichia broth expressing factor VIII:C heavy chain concentrated by 0→80% saturation ammonium sulphate) is then injected, followed by which the column is washed with equilibration buffer (pH 6.0) until the absorbance at 280 nm reaches baseline. The said wash step is carried out to eliminate weakly bound impurities that would otherwise co-elute along with the heavy chain. Elution is carried out by lowering the pH (protonation), using 20 mM-100 mM of Acetate buffers containing 0.5 M NaCl (pH 4.0-6.0), preferably 50 mM acetate buffers containing 0.5M NaCl at pHs 6.0, 5.0 and 4.0 respectively. This elution technique is advantageous over the alternate imidazole-based elution methods, as imidazole is toxic and would require additional desalting steps when considering the purified protein for therapeutic applications.

After the chromatographic runs, the columns are stripped off with metal ions by using ethylene diamine tetra acetate (EDTA) and the columns are regenerated with 50 mM NaOH.

SDS-PAGE, followed by western blotting is performed on purified samples to confirm the presence of factor VIII:C heavy chain. About 5 μg of obtained protein sample is loaded on each well of a polyacrylamide gel (10% polyacrylamide gel), and SDS-PAGE is performed. After electrophoresis, the proteins from the polyacrylamide gel are transferred to a nitrocellulose membrane using Mini Trans-Blot Cell (BIO-RAD, India). Towbin transfer buffer (25 mM Tris, 200 mM glycine, 0.01% SDS, 20% methanol, pH 8.3) is used to carry out the transfer from the polyacrylamide gel to the nitrocellulose membrane, at 90V for about 90 mins at a temperature of about 4° C. The membrane is washed with PBST, blocked with a blocking buffer (PBST with 5% skimmed milk powder) overnight at a temperature of about 4° C. After blocking, the membrane is incubated with primary antibody (rabbit anti-A1 or anti-C2 antiserum, along with excess of control Pichia broth to prevent non-specific binding) at room temperature for about one hour. The membrane is washed about 2-4 times with PBST solution after which it is incubated with secondary antibody (goat anti-rabbit IgG, HRP conjugated) at room temperature for about 1 hour. The membrane is then washed for about 2-4 times with PBST. The membrane is developed by diaminobenzidine/H2O2 method for detection of HRP.

The rabbit anti-A1 antiserum used for the detection of the expressed heavy chain protein by western blotting is prepared using E. coli expressed heavy chain terminal domain A1 domain, tagged with GST as antigen. Rabbits are immunized with the purified antigen (A1-GST). During addition of rabbit antisera to the membrane, the antiserum is incubated along with excess of Pichia pastoris control broth to reduce non-specific binding and background noise.

Purification Results

Immobilized metal-ion affinity chromatography (IMAC) is employed to purify the expressed heavy chain of FVIII:C in Pichia pastoris clones. The rationale behind choosing IMAC is the presence of several Histidine residues on the surface of the heavy chain molecule which is confirmed based on the crystal structure of factor VIII:C.

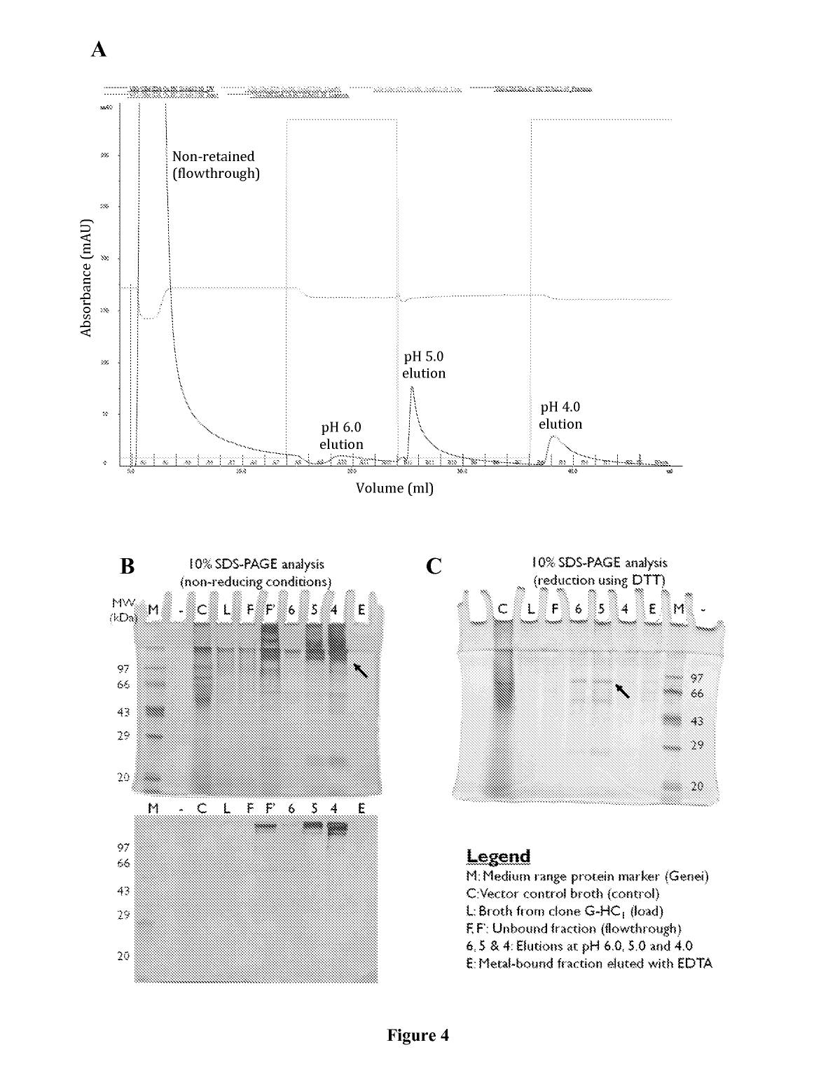

The integrity of the purified protein is analysed over SDS-PAGE and western blotting. While sharp peaks are observed during each elution step (FIG. 4A), it is observed that the heavy chain protein is obtained to near homogeneity upon elution at pH 5.0. When analysed by SDS-PAGE and western blotting under non-reducing conditions (FIG. 4B), the heavy chain protein is sometimes observed to aggregate as a dimer, which breaks down upon reduction with DTT showing a clear band at the expected 90 kDa molecular weight (FIG. 4C, lane 5).

This purified factor VIII:C heavy chain is used for reconstitution with purified light chain to further assess the coagulation activity.

### EXAMPLE 4

Activity Results of Complete Factor VIII:C Protein Reconstituted from Individual Light Chain and Heavy Chain

Full length B-domain deleted Factor VIII:C is regenerated from individual heavy & light chains expressed in Pichia pastoris expression system. Equimolar concentrations of purified heavy and light chains are reconstituted in 20 mM HEPES, pH 7.0-7.4 containing 0.3M KCl, 0.01% Tween-20, 0.01% BSA, 25 mM CaCl₂ and 0.5 μM Cu⁺⁺ and is incubated at a temperature of about 20° C.-23° C. for a time-period of about 4 hours-6 hours. Following this, the coagulation activity of the reconstituted FVIII is determined by the one-stage clotting assay using FVIII-depleted plasma (Siemens Healthcare Diagnostics, USA) over IL ACL 10000 coagulation analyser (Instrumentation Laboratory, Italy). The One stage clotting assay is performed as follows

Factor VIII-depleted plasma is dissolved in distilled or deionized water. Before use, it is allowed to stand for at least 15 minutes at a temperature of about 15 to 25° C., and then mixed carefully without foam formation. The reconstituted recombinant Factor VIII:C sample is added to FVIII deficient plasma. APTT reagents are used according to the manufacturer's instructions. 0.025M of CaCl₂ is added to the mixture. The mixture is incubated at a temperature of about 37° C. and for a time-period of about 2 minutes and the reading is taken in an automated coagulation analyzer. Normal clotting time is 30-40 seconds.

The specific activity of the reconstituted recombinant FVIII:C [Light chain and Heavy chain purified using HLAC and IMAC systems respectively] is found to be 7665 IU/mg, as determined by the chromogenic assay.

### EXAMPLE 5

Purification of Recombinant FVIII Expressed in CHO Cells Using HLAC

Recombinant BDD (B-domain deleted)-FVIII [rBDD-FVIII] or functionally active FVIII expressed in CHO cell lines is purified using histidine affinity chromatography.

5 ml of harvested CHO cell culture supernatant (CSS) containing rBDD-FVIII is directly passed over a CIM-Histidine disk, pre-equilibrated with 20 mM Tris, pH 6.0. After binding, the column is thoroughly washed with the same buffer, post which elution is carried out using 20 mM Tris, pH 7.0 containing Glycine, Lysine, and CaCl₂.

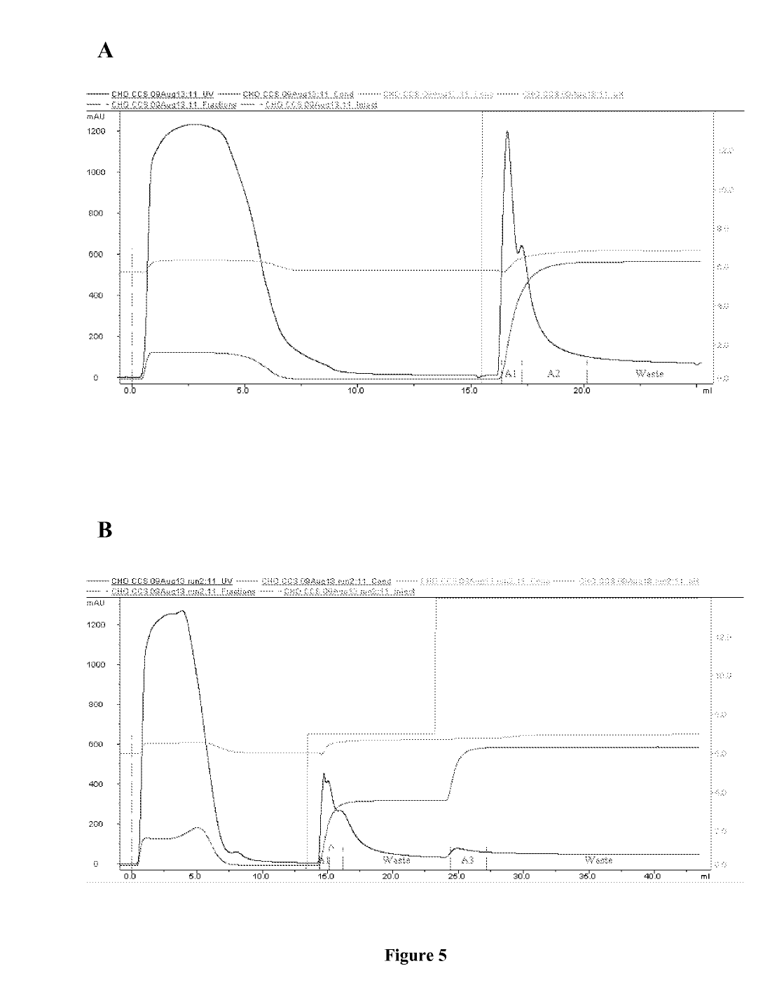

The chromatographic profile of this purification run is depicted in FIG. 5A. Two overlapping peaks are obtained (fractions A1 and A2), which upon activity analysis reveal that the factor VIII activity is found on the latter fraction (Table 4). The purification and activity results showcase a 1567 fold increase in purity of rBDD-FVIII.

TABLE 4
Activity table for purification of rBDD-VIII from
CHO cell lines (without intermediary wash step)

| | Amount | Specific Activity |
|---|---|---|
| Fraction | (mg) | (IU/mg) |
| Load | 13.32 | 0.23 |
| FT | 10.24 | 0.20 |
| Peak (A1) | 1.10 | 0.81 |
| Tail (A2) | 0.78 | 360.29 |

Further, an intermediary wash step (involving a mix of 50% binding buffer and 50% elution buffer) is introduced, in order to separate the two bands (A1 and A2) obtained above. The purification profile of the protocol having an intermediary wash step is depicted in FIG. 5B. It is observed that a better separation is obtained with the introduction of intermediary wash step, resulting in a higher specific activity of the purified fraction (A3) [Table 5]. The results showcase a 6179-fold increase in purity of rBDD-FVIII.

TABLE 5
Activity table for purification of rBDD-VIII from
CHO cell lines (with intermediary wash step)

| | Amount | Specific Activity |
|---|---|---|
| Fraction | (mg) | (IU/mg) |
| Load | 13.24 | 0.23 |
| FT | 10.44 | 0.19 |
| 50% B peak (A1-A2) | 1.13 | 8.28 |
| 100% B peak (A3) | 0.13 | 1421.28 |

While preferred embodiments have been shown and described in the present disclosure, various modifications and substitutions may be made thereto without departing from the spirit and scope of the present invention. Accordingly, it is to be understood that apart from sources such as plasma, liver, CHO cells, Pichia pastoris, the presently disclosed HLAC and IMAC purification methods can be successfully employed to purify FVIII from various natural and recombinant sources including but not limiting to Lemna gibba and Hansenula polymorpha. The said purification aspects are well within the scope of the present disclosure.

### EXAMPLE 7

Comparative Study of the Efficiency of Pseudobioaffinity Methods of the Present Disclosure and Currently Known Purification Methods

TABLE 6
Purification of FVIII from plasma/cryoprecipitate

| | | | | Specific | | |
|---|---|---|---|---|---|---|
| | | | | Activity of | | |
| Purification | Protein | | | product | | Purification |
| method | Purified | Source | No. of steps | (IU/mg) | Recovery % | Fold |
| CIM-His [HLAC] | Full-length | Plasma | One | 693 | 55% | 1386 |
| [Present Invention] | Factor VIII | Cryoprecipitate | | | | |
| Aluminum adsorption, | Full-length | Plasma | Six | 175.4 ± 37.8 | 55-65% | — |
| cold precipitation | Factor VIII | Cryoprecipitate | | | | |
| and Ion Exchange | | | | | | |
| chromatography | | | | | | |
| [Prior Art Method] | | | | | | |
| Anion exchange | Full-length | Plasma | Two | — | 40% | 54 |
| chromatography using | Factor VIII | | | | | |
| Q-Sepharose XL | | | | | | |
| [Prior Art Method] | | | | | | |

TABLE 7
Purification of FVIII and FVIII individual chains (heavy and light chains) from recombinant sources

| | | | | Specific Activity | |
|---|---|---|---|---|---|
| Purification | Protein | | | of product | Purification |
| method | Purified | Source | No. of steps | (IU/mg) | Fold |
| CIM-His [HLAC] | Recombinant | CHO cell culture | One | 1421.28 | 6179 |
| [Present Invention] | BDD-Factor | supernatant | | | |
| | VIII | | | | |
| CIM-His [HLAC] | FVIII Light | Pichia pastoris | One | 7665 (upon | — |
| [Present Invention] | Chain | | | reconstitution) | |
| CIM-Cu⁺⁺ [IMAC] | FVIII Heavy | Pichia pastoris | One | 7665 (upon | — |
| [Present Invention] | Chain | | | reconstitution) | |
| Mixed mode chromatography | Recombinant | CHO cell culture | One (only capture; | 5300 | 96 |
| using CaptoMMC | Factor VIII | supernatant | further polishing | | |
| | | | required) | | |
| Affinity chromatography | Recombinant | — | Two (only capture; | — | — |
| (VIIIselect - 13 kDa | Factor VIII | | further polishing | | |
| recombinant peptide) | | | required) | | |
| [Prior Art Method] | | | | | |
| Affinity chromatography | Simulated | Pre-purified | One (only capture; | — | 63 |
| (synthetic ligand L4) | recombinant | plasma-derived | further polishing | | |
| [Prior Art Method] | Factor VIII | FVIII in cell | required) | | |
| | | culture supernatant | | | |
| Mixed-mode (capto MMC), | Recombinant | HEK | Eight | — | — |
| cation exchange, filtration, | Factor VIII | | | | |
| affinity (VIIIselect), anion | | | | | |
| exchange chromatography | | | | | |
| [Prior Art Method] | | | | | |
| Affinity chromatography | FVIII Heavy | Baculovirus- | One | 0.0285 | — |
| (using anti-HC & | Chain and | insect cell | | | |
| anti-LC antibodies) | Light Chain | (Sf9) | | | |
| [Prior Art Method] | | system | | | |

Note:
The number of purification steps signifies the number of purification processes involved, and not the intermediary wash steps that are part of a particular chromatographic method.

The present disclosure thus discloses successful purification of Factor VIII protein and/or its fragments from a number of sources by employing various pseudobioaffinity based purification methods with minimal steps. Histidine Ligand Affinity Chromatography (HLAC) is used for the purification of full-length Factor VIII from plasma cryoprecipitate, for the purification of recombinant B-domain deleted Factor VIII:C expressed in various recombinant host systems and for the purification of recombinant factor VIII light chain expressed in Pichia pastoris. Similarly, Immobilized metal-ion affinity chromatography (IMAC) is employed for the purification of recombinant factor VIII:C heavy chain expressed in Pichia pastoris, for the purification of recombinant B-domain deleted Factor VIII:C expressed in various recombinant host systems etc. More importantly, the purification efficiency showcased by the methods of the present disclosure is far superior when compared to the presently available methods for the purification of factor VIII and hence the presently disclosed methods provide better alternatives for therapeutic applications involving factor VIII (such as treatment of hemophilia). Thus, the purification methods of the present disclosure successfully overcome the hurdles associated with FVIII purification in prior art and act as immense potential applications in the pharma industry to purify Factor VIII from various natural and recombinant sources.

1.-21. (canceled)

22. A method of purifying Factor VIII protein or fragment thereof from a sample, said method comprising act of subjecting the sample to monolith based pseudo-bioaffinity purification to obtain said purified Factor VIII protein or fragment thereof, wherein the monolith based pseudo-bioaffinity purification comprises act of coupling or immobilizing a monolith column with a ligand selected from a group consisting of L-histidine and chelated transition metal ion.

23. The method as claimed in claim 22, wherein the Factor VIII protein or fragment thereof within the sample is naturally occurring or recombinant Factor VIII protein or fragment thereof, or a combination thereof.

24. The method as claimed in claim 23, wherein the natural Factor VIII protein or fragment thereof is obtained from a source selected from a group comprising plasma and liver or a combination thereof; or the recombinant Factor VIII protein or fragment thereof is obtained from a cell selected from a group comprising CHO cell, Pichia pastoris, Lemna gibba and Hansunella polymorpha, or any combination thereof.

25. The method as claimed in claim 22, wherein the Factor VIII fragment is Factor VIII Light Chain, Factor VIII heavy chain, or a combination thereof.

26. The method as claimed in claim 25, wherein the Factor VIII heavy chain has a molecular weight ranging from about 90 kDa to 210 kDa; or wherein light chain has a molecular weight of about 80 kDa.

27. The method as claimed in claim 22, wherein the method purifies Factor VIII protein or fragment thereof with purification factor value ranging from about 295 fold to about 6179 fold.

28. The method as claimed in claim 22, wherein the yield of purified Factor VIII protein or fragment thereof is at least about 55%.

29. The method as claimed in claim 22, wherein the monolith based pseudo-bioaffinity purification comprises acts of:
a) coupling or immobilizing a monolith column with a ligand selected from a group consisting of L-histidine and chelated transition metal ion to obtain an immobilized pseudobioaffinity monolith column;
b) equilibrating the immobilized monolith column obtained in step (a) and loading the sample into the column;
c) optionally carrying out a wash step; and
d) eluting the sample to obtain the Factor VIII protein or fragment thereof.

30. The method as claimed in claim 29, wherein the monolith based pseudo-bioaffinity purification is Histidine Ligand Affinity Chromatography (HLAC) when the ligand is L-histidine; or wherein the monolith based pseudo-bioaffinity purification is Immobilized metal-ion affinity chromatography (IMAC) when the ligand is chelated transition metal ion.

31. The method as claimed in claim 29, wherein the transition metal ion is selected from a group comprising copper, nickel, cobalt and zinc, or any combination thereof.

32. The method as claimed in claim 29, wherein the monolith column is Convective Interaction Media (CIM) monolithic column.

33. The method as claimed in claim 29, wherein the coupling or immobilization is carried out in presence of coupling agent selected from a group comprising ethylene-di-amine (EDA), carbonyldiimidazole (CDI), epoxy, imino-di-acetic acid (IDA) and Tris(2-aminoethyl)amine (TREN), or any combination thereof.

34. The method as claimed in claim 29, wherein the equilibration is carried out by employing buffer selected from a group comprising cationic buffer, phosphate buffer, 3-(N-morpholino)propanesulfonic acid (MOPS) buffer and MMA buffer, or any combination thereof and wherein said cationic buffer is selected from a group comprising Tris-HCl, and bicarbonate, or a combination thereof.

35. The method as claimed in claim 34, wherein concentration of the cationic buffer ranges from about 20 mM to about 100 mM and pH ranges from about 5.5 to about 6.5; and wherein concentration of the phosphate buffer, MOPS buffer and MMA buffer ranges from about 20 mM to about 100 mM and pH ranges from about 7.0 to about 8.0.

36. The method as claimed in claim 34, wherein the phosphate buffer or MOPS buffer or MMA buffer contain salt at concentration ranging from about 0.5 M to about 2 M.

37. The method as claimed in claim 29, wherein the elution is carried out by employing buffer selected from a group comprising cationic buffer containing calcium (II) ions, glycinate ions and lysine, acetate buffer containing sodium chloride, or a combination thereof; and wherein the cationic buffer is selected from a group comprising Tris-HCl, and bicarbonate, or a combination thereof.

38. The method as claimed in claim 37, wherein concentration of the cationic buffer ranges from about 20 mM to about 100 mM and pH ranges from about 7 to about 8; and wherein concentration of the acetate buffer ranges from about 20 mM to about 100 mM and pH ranges from about 4 to about 5.

39. The method as claimed in claim 29, wherein the wash step is carried out by employing buffer selected from a group comprising cationic buffer, phosphate buffer, 3-(N-morpholino)propanesulfonic acid (MOPS) buffer and MMA buffer, or a combination thereof; and wherein the cationic buffer is selected from a group comprising Tris-HCl, and bicarbonate, or a combination thereof.

40. The method as claimed in claim 39, wherein concentration of the cationic buffer ranges from about 10 mM to about 50 mM and pH ranges from about 7 to about 8; and wherein concentration of the phosphate buffer, 3-(N-morpholino)propanesulfonic acid (MOPS) buffer and MMA buffer ranges from about 20 mM to about 100 mM and pH ranges from about 6.0 to about 7.0.

41. A purified, biologically active Factor VIII protein or fragment thereof obtained according to the method of claim 1, wherein the method purifies Factor VIII protein or fragment thereof with purification factor value ranging from about 295 fold to about 6179 fold.
